(19) United States
(12) Patent Application Publication
(10) Pub. No.: US 2006/0069373 A1
(43) Pub. Date: Mar. 30, 2006

(54) Ultrasonic attachment of an elastic strand

(75) Inventors: Daniel R. Schlinz, Greenville, WI (US); John F. Condron, Neenah, WI (US); Thomas D. Ehlert, Neenah, WI (US); James M. Gaestel, Seymour, WI (US); Davis-Dang H. Nhan, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/957,152

(22) Filed: Sep. 30, 2004

(51) Int. Cl. A61F 13/15

(52) U.S. Cl. 604/385.3; 604/385.31

(57) ABSTRACT

Unique elastic composites suitable for a variety of uses including but not limited to disposable absorbent articles. One version of the elastic composite includes a first layer, a second layer and at least one elastic strand located between the first layer and the second layer, the elastic strand defining a width.

### BACKGROUND

The present invention relates to improved elastic composites. More particularly, the present invention relates to absorbent articles containing improved elastic composites.

Various elastic composites are known for use in connection with absorbent articles such as infant diapers, training pants, adult incontinence products and the like. Such elastic composites are generally employed along the longitudinal side edges or lateral end edges of an absorbent article such as a diaper. The elastic composites are generally thought to be particularly well suited for improving the fit and containment of the absorbent article.

Elastic composites are often used in connection with elasticized leg cuffs located at the leg openings of the absorbent article which leg cuffs perform an additional gasketing function.

Many constructions of elastic composites are known. Generally, an elastic material is contained between two opposing webs. The webs are joined together forming a tube or channel containing the elastic material. Further, the elastic material is attached in some manner to one or both of the opposing webs such that the elastic material gathers the elastic composite.

In order to allow the elastic material to gather the elastic composite, the elastic material is generally adhesively attached to the elastic composite. Adhesive attachment of the elastic material to the elastic composite results in a certain amount of the elastic nature of the elastic material being destroyed. Specifically, at the points where the elastic material is adhesively attached to the elastic composite, the elastic material is no longer capable of being stretched. That is, it is no longer “elastic”. Moreover, the use of adhesives to attach the elastic material, or in joining the two layers of material, has been found, in some instances, to undesirably affect the stiffness of the elastic composite.

Several ultrasonic techniques have been used to attach the elastic material to the opposing webs. The first technique utilizes a solid bar that bonds completely over the elastic material as well as the opposing web on both sides of the elastic material. A second technique elongates the elastic material, causing it to narrow, and then bonds the opposing webs together forming a tube slightly larger than the narrowed elastic. When the elastic material relaxes, it swells and becomes “trapped” in the tube. A third technique utilizes an elastic “band” which is relatively wide where the opposing webs are bonded together through the elastic “band”.

Each of the above techniques present drawbacks in either the processability of the composite, the type of elastic material the can be utilized, or the attributes of the finished elastic composite. As a result, there has remained a need to provide an improved elastic composite.

### SUMMARY

In response to the foregoing need, the present inventors undertook intensive research and development efforts that resulted in the discovery of unique elastic composites suitable for a variety of uses including disposable absorbent articles. One version of the elastic composite of the present invention includes a first layer, a second layer and at least one elastic strand located between the first layer and the second layer, the elastic strand defining a width. Additionally, the elastic composite includes a plurality of thermoplastic bonds joining the first and second layers, at least a portion of the thermoplastic bonds containing a segment of the elastic strand. The portions have a spacing perpendicular to the elastic strand that is less than the width of the elastic strand.

In another version, a disposable absorbent article includes an absorbent core, an outer cover positioned adjacent the absorbent core and a pair of containment flaps extending at least partially along the length of the absorbent core. The containment flaps include a first layer, a second layer and at least one elastic strand located between the first layer and the second layer, the elastic strand defining a width. Additionally, the containment flaps include a plurality of thermoplastic bonds joining the first and second layers, at least a portion of the thermoplastic bonds containing a segment of the elastic strand. The portions have a spacing perpendicular to the elastic strand that is less than the width of the elastic strand.

In another version, an elastic composite includes a first layer, a second layer and at least one elastic strand located between the first and second layers. Additionally, the elastic composite includes a plurality of thermoplastic bonds joining the first layer and the second layer; wherein portions of the elastic strand are contained in at least a portions of the thermoplastic bonds. The elastic strand has a width. The thermoplastic bonds containing the elastic strand have a spacing in a direction perpendicular to the elastic strand that is between 0.1 and 0.9 times the width of the elastic strand. The thermoplastic bonds containing the elastic strand have a dimension perpendicular to the elastic strand that is between 0.2 and 0.6 times the width of the elastic strand. The elastic strand has a cross sectional shape of a circle. The thermoplastic bonds intermittently bond the elastic strand to the first and second layers. The elastic strand has a decitex of less than 2000.

One version of the present invention provides a method of producing an elastic composite. The method includes providing a first layer, providing a second layer, and providing at least one elastic strand located between the first layer and the second layer and defining a width. The method also includes joining the first and second layers with a plurality of thermoplastic bonds, at least a portion of the thermoplastic bonds containing a segment of the elastic strand. The portions have a spacing perpendicular to the elastic strand that is less than the width of the elastic strand.

### DRAWINGS

The foregoing and other features and aspects of the present invention and the manner of attaining them will become more apparent, and the invention itself will be better understood by reference to the following description, appended claims and accompanying drawings, where:

FIG. 1 illustrates a version of the elastic composite, with portions broken away for purposes of illustration;

FIG. 2 illustrates a version of the elastic composite that contains two elastic strands;

FIG. 3 illustrates an enlarged view of the elastic composite showing details of the thermoplastic bonds;

FIG. 4 illustrates an enlarged cross section of an elastic composite;

FIG. 5 illustrates a version of the elastic composite showing details of the thermoplastic bonds; and

FIG. 6 illustrates an apparatus capable of producing an elastic composite;

FIG. 7 illustrates a plan view of a disposable absorbent article in an unfolded, flat-out, uncontracted state (i.e., with all elastic induced gathering and contraction removed), with the bodyfacing surface of the article facing the viewer and with portions of the article partially cut away to illustrate underlying features.

### DESCRIPTION

The present invention relates to elastic composites (16), illustrative embodiments of which are shown in the Figures. The elastic composites (16) are suitable for incorporation into a variety of disposable absorbent articles.

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

All percentages, ratios and proportions used herein are by weight unless otherwise specified.

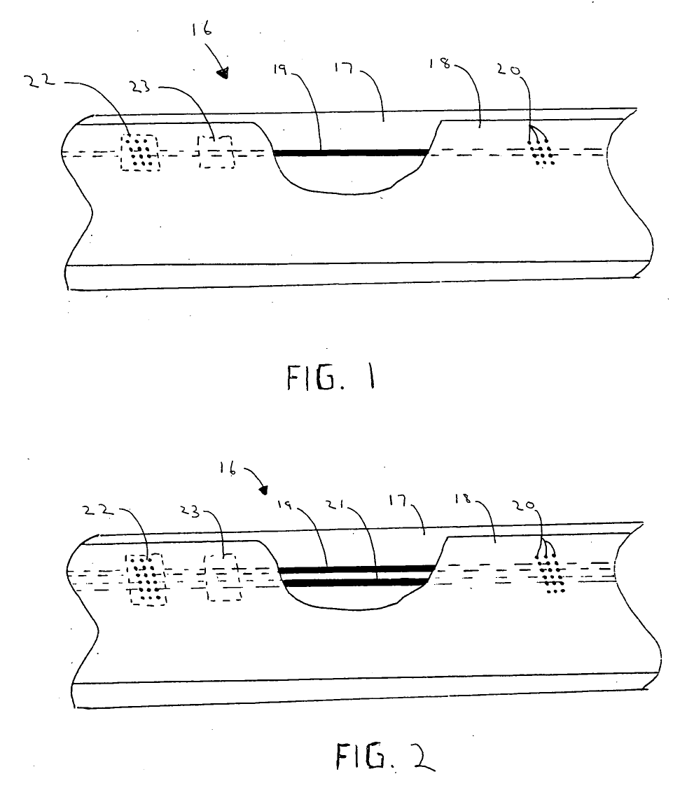

As illustrated in FIG. 1, the elastic composite (16) of the present invention includes a first layer (17) of fusible material, a second layer (18) of fusible material, and at least one elastic strand (19). Further, the elastic composite (16) of the present invention includes a plurality of thermoplastic bonds (20). Some of the thermoplastic bonds (20) contain at least a portion of the elastic strand (19). As used herein, a thermoplastic bond “contains a portion of an elastic strand” when at least some of the material that makes up the elastic strand has been integrated in the thermoplastic bond. It is not necessary for the material of the elastic strand to be meltable. One method to determine if a thermoplastic bond contains a portion of an elastic strand is to preferentially stain the elastic strand and then view the composite normal to the surface using a suitable microscope.

A wide range of materials is suitable for use as the first (17) and second (18) layers. A material will be considered fusible when an applied energy causes it to soften, melt or flow such that it can be joined to another material. As used herein, reference to “thermoplastic bonds” and the like refer to bonding caused by the application of energy, such as heat, ultrasonic energy, infrared energy, pressure and the like.

Specific examples of materials suitable for use as the first (17) and second layers (18) of fusible material include nonwoven materials, such as spunbond or meltblown thermoplastic polymers, such as polyolefins; bonded carded webs; film materials, such as polyolefin, ethylene vinyl acetate, ethyl methacrylate, and polyester films; foam materials, such as polyolefin foams; woven materials, such as woven polypropylene, polyethylene or polyester fabrics; and composites and laminates of the above nonwoven, film, foam, and woven materials. In a specific embodiment of the present invention, such as that illustrated in FIG. 1, the first (17) and second (18) fusible materials are non-integrally formed. That is, the first (17) and second (18) layers of fusible material represent separate elements that are not joined other than by thermal, adhesive or similar attaching techniques. Specifically, the first (17) and second (18) layers are not formed from an integral piece of material through a folding process. In another specific embodiment of the present invention, such as that illustrated in FIG. 6, the first (17) and second (18) fusible layers are integrally formed. That is, the first (17) and second (18) layers are formed from a single, integral piece of material through a folding process.

The first layer (17) and/or the second layer (18) may be stretchable, either elastically or extensible. In particular embodiments, either or both the first layer (17) and the second layer (18) can provide a stretch elongation that is no less than about 20%, alternatively, no less than about 60%; and finally, alternatively, no less than about 100%. In particular embodiments, the first layer (17) or the second layer (18) can provide a stretch elongation measured at 250 g/in that are no more than about 120%; alternatively, no more than about 100%; and finally, alternatively, no more than about 50%. Thus, the first layer (17) or the second layer (18) typically can provide a stretch elongation measured at 250 g/in that is no less than about 20% and no more than about 120%; although the approximate percent may vary according to, inter alia, the general design and intended use of the elastic composite (16). Further, the stretch elongation of the first layer (17) may be different than the stretch elongation of the second layer (18).

In a specific embodiment, the first (17) and second (18) layers of fusible materials are formed from a nonwoven material such as a spunbond or meltblown polyethylene or polypropylene material. When it is desired to provide an elastic composite (16) which is generally liquid pervious, the spunbond material will suitably be treated with a surfactant to render it generally hydrophilic. Alternatively, one of the first (17) or second (18) layers of fusible material may be treated with a surfactant while the other layer is treated to a lesser extent with a surfactant or is not treated with a surfactant. In this case, an elastic composite that is less pervious to liquid will be produced. Still further, if it desired to produce an elastic composite that is generally liquid impervious, one of the first (17) or second (18) layers of fusible material can comprise a liquid-impervious film, such as a polyolefin film, or the first (17) or second (18) layers of fusible material can comprise a spunbond-meltblown-spunbond or other suitably liquid-impervious nonwoven.

In another specific embodiment, the first (17) layer of fusible material is a SMS material having a basis weight of about 19 grams per square meter. The second layer of fusible material comprises a SMS material formed from polypropylene and having a basis weight of about 19 grams per square meter and comprising about 75 percent spunbond material and about 25 percent meltblown material.

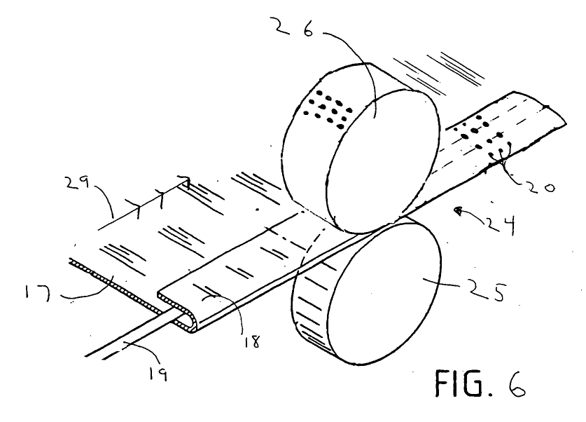

The elastic strand (19) may comprise any elastomeric strand capable of being elongated at least about 50 percent, alternately at least about 250 percent, alternately at least about 350 percent, and capable of recovering to a length within at least about 75 percent, more particularly, at least about 50 percent of its elongated length (original length plus elongation). The elastic strand (19) may have a variety of cross sectional shapes, including but not limited to a circle, oval, triangle or square. The elastic strand (19) may have a width (C) and a height (D) as shown in FIG. 4. To be considered a strand the elastic should have a width to height ratio (C/D) of less than about 3.

The elastic strand (19) may be in the form of individual elastomeric threads of elastomeric material which are spun together to form the elastic strand (19). In some embodiments, the elastic strand (19) may consist of 30 or more elastic threads. In one specific embodiment, the elastic stand (19) may be a 1520 decitex elastomeric thread commercially available under the tradename LYCRA from INVISTA of Wichita, Kans. Alternatively, the elastic strand (19) may be composed of a thermoplastic elastomer commercially available under the tradename KRATON from Krayton Polymers of Houston, Tex. or a natural or synthetic rubber commercially available from J.P.S. Elastomerics Corp. of Holyoke, Mass. The elastic strand (19) may also be composed of a heat-activatable elastic material such as Pebax®, commercially available from Atochem, Inc. of Philadelphia, Pa., which can be activated with heat treatment after the association with the elastic composite (16). The elastic strand may have a decitex that is less than 5000, alternatively less than 2000 and finally less than 1000 decitex.

The elastic composite (16) may comprise a single, elastic strand or a plurality of elastic strands, for example, a first elastic strand (19) and second elastic strand (21) as illustrated in FIG. 2.

In the embodiments illustrated in FIGS. 1 and 2, thermoplastic bonds (20) intermittently attach the first (17) and second (18) layer of fusible material. The intermittent thermoplastic bonds (20) define areas containing the thermoplastic bonds (22) and areas remote from the thermoplastic bonds (23), both areas (22, 23) containing at least the first (17) and second (18) layers of fusible material and the elastic strand/s (19, 21).

The areas containing the thermoplastic bonds (22) may have a basis weight that is greater than the basis weight in the area remote from the thermoplastic bonds (23). The higher basis weight in the area containing the thermoplastic bonds (22) provides for more material to participate in the thermoplastic bonds (20) and thereby creates stronger bonds. The lower basis weight in the areas remote from the thermoplastic bonds (23) allows the elastic strand (19) to gather to a higher degree. This difference in basis weight may be achieved in a number of ways. For example, an additional layer of material may be cut and placed into the area containing the thermoplastic bond (22) prior to bonding. Alternatively, the basis weight of the individual layers (17, 18) of fusible material may greater in the area containing the thermoplastic bonds (22) than in the area remote from the thermoplastic bonds (23). Still alternatively, additional thermoplastic material may be added to the areas containing the thermoplastic bonds (22) by either spraying, printing, slot coating or other suitable methods. This additional thermoplastic material may be an adhesive. Preferably, the additional thermoplastic material may be polyethylene, polypropylene, formulated polypropylene, formulated polyethylene, or other suitable non-adhesive thermoplastic material. The use of a non-adhesive prevents the outer surfaces or edges of the elastic composite (16) from becoming tacky or sticky.

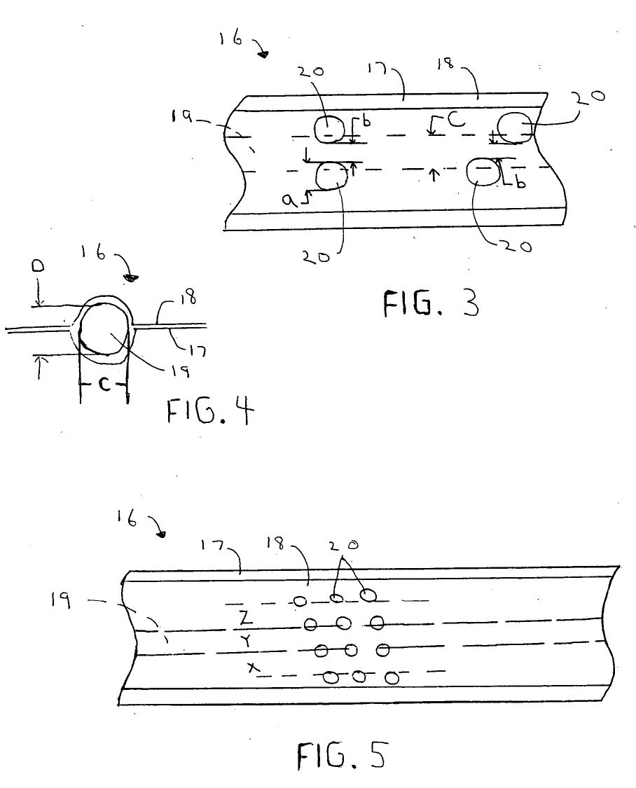

FIG. 3 illustrates the relative size and spacing of the thermoplastic bonds (20) in relations to the elastic strand (19). The thermoplastic bonds (20) may have a dimension perpendicular to the elastic strand (a) that is less than the width of the elastic strand (c). This relationship allows for a broader operating window during manufacture. During manufacture, a variation in the basis weight of the first (17) or second (18) layer, the thickness of the elastic strand (19) or the intensity of the energy forming the thermoplastic bond (20) may cause the thermoplastic bond (19) to cut through the elastic strand (19). If the thermoplastic bond (20) extends across the width (c) of the elastic strand (19) this cutting would likely disrupt the process, further, the elastic strand (19) would likely not be attached strongly enough to the first (17) and second (18) layers of fusible material to remain intact during use. By providing a thermoplastic bond (20) with a dimension perpendicular to the elastic strand (a) that is less than the width of the elastic strand (c), even if conditions exist that would cut through the elastic strand (20) in the area of the thermoplastic bond (20) at least a portion of the elastic strand (19) would remain intact.

The thermoplastic bonds (20) may have a dimension perpendicular to the elastic strand (a) that is less than 0.9 times the width of the elastic strand (c). Alternatively, the thermoplastic bonds (20) may have a dimension perpendicular to the elastic strand (a) that is less than 0.6 times the width of the elastic strand (c). Alternatively, the thermoplastic bonds (20) may have a dimension perpendicular to the elastic strand (a) that is less than 0.3 times the width of the elastic strand (c). The thermoplastic bonds (20) may have a dimension perpendicular to the elastic strand (a) that is greater than 0.15 times the width of the elastic strand (c). Alternatively, the thermoplastic bonds (20) may have a dimension perpendicular to the elastic strand (a) that is greater than 0.2 times the width of the elastic strand (c). Alternatively, the thermoplastic bonds (20) may have a dimension perpendicular to the elastic strand (a) that is greater than 0.4 times the width of the elastic strand (c). In specific embodiments, the thermoplastic bonds (20) may have a dimension perpendicular to the elastic strand (a) less than 0.050 in. (1.27 mm), alternatively less than 0.025 in. (0.64 mm), alternatively, less than 0.010 in. (0.25 mm). In specific embodiments, the thermoplastic bonds (20) may have a dimension perpendicular to the elastic strand (a) greater than 0.005 in (0.132 mm), alternatively greater than 0.025 in. (0.64 mm), alternatively, greater than 0.040 in. (1.02 mm).

The thermoplastic bonds (20) containing the elastic strand (19) may have a inter-bond spacing (b) in the direction perpendicular to the elastic strand (19) that is less than the width of the elastic strand (c), shown in FIG. 3. This spacing assists in the thermoplastic bonds (20) containing the elastic stand (19). The thermoplastic bonds (20) containing the elastic strand (19) may have a spacing (b) in the direction perpendicular to the elastic strand (19) that is less than 0.9 times the width of the elastic strand (c). The thermoplastic bonds (20) containing the elastic strand (19) may have a spacing (b) in the direction perpendicular to the elastic strand (19) that is less than 0.5 times the width of the elastic strand (c). The thermoplastic bonds (20) containing the elastic strand (19) may have a spacing (b) in the direction perpendicular to the elastic strand (19) that is less than 0.25 times the width of the elastic strand (c). The thermoplastic bonds (20) containing the elastic strand (19) may have a spacing (b) in the direction perpendicular to the elastic strand (19) that is greater than 0.1 times the width of the elastic strand (c). The thermoplastic bonds (20) containing the elastic strand (19) may have a spacing (b) in the direction perpendicular to the elastic strand (19) that is greater than 0.25 times the width of the elastic strand (c).

The thermoplastic bonds (20) containing the elastic strand (19) may have a spacing (b) in the direction perpendicular to the elastic strand (19) that is greater than 0.5 times the width of the elastic strand (c). In specific embodiments, the thermoplastic bonds (20) containing the elastic strand (19) may have a spacing (b) in the direction perpendicular to the elastic strand (19) that is greater than 0.005 in (0.132 mm), alternatively greater than 0.010 in. (0.25 mm), alternatively, greater than 0.040 in. (1.02 mm). In specific embodiments, the thermoplastic bonds (20) containing the elastic strand (19) may have a spacing (b) in the direction perpendicular to the elastic strand (19) that is less than 0.050 in (1.27 mm), alternatively less than 0.025 in. (0.64 mm), alternatively, less than 0.010 in. (0.25 mm).

The thermoplastic bonds (20) may be arranged in substantially parallel rows to create a plurality of paths through which an elastic strand (20) may pass. As illustrated in FIG. 5, the thermoplastic bonds (20) are arranged in four substantially parallel rows creating three paths (X, Y, Z) through which an elastic strand (20) may pass. As illustrated, an elastic strand (20) passes through the center path (Y). These additional paths, along with the thermoplastic bond (20) spacing and size provide several processing benefits. During processing, if an elastic strand (20) weaves from side to side, the elastic composite (20) will remain functional. The spacing between the thermoplastic bonds (20) insures that the elastic strand (19) is securely held between the first (17) and second (18) layers of fusible material. The dimension of the thermoplastic bonds (20) perpendicular to the elastic strand (19) insures that if the thermoplastic bond (20) does partially cut through the elastic strand (19), the elastic strand (19) will remain intact. Further, the plurality of paths allows the elastic strand (19) to weave in the process and remain anchored in the thermoplastic bonds (20).

The present invention provides methods of making an elastic composite (20) as described above. One method can best be understood by reference to FIG. 6 wherein a first material is provided from supply roll (not shown). The first material forms the first layer of heat-fusible material (17) and travels in a direction as indicated by the arrow (29). An elastic strand (19) is supplied from supply roll (not shown), is elongated and travels in the direction as indicated. The first material is folded around the elastic strand (19) forming the second layer (18). The composite then passes through a thermal bonding means such as an ultrasonic bonder (24). The sonic bonder (24) thermally bonds the first layer (17) to the second layer (18) of fusible material through thermal bonds (20). The sonic bonder (24) consists of an anvil roll (25) and a pattern roll (26). The pattern roll (26) contains raised elements which, when compressing the first layer (17), second layer (18) and elastic strand (19) form thermoplastic bonds (20) thereby forming the elastic composite (20).

The various aspects, benefits, and versions of the elastic composite (16) will be described in the context of a disposable absorbent article, such as a disposable diaper. It is, however, readily apparent that one or more versions of the present invention could also be employed with other disposable absorbent articles, such as feminine hygiene articles, children's training pants, adult incontinence garments, surgical gowns and the like. Further, the elastic composite (16) may be employed in any application where elastic properties are desired, such as durable garments and sporting goods. Typically, disposable absorbent articles are intended for limited use and are not intended to be laundered or otherwise cleaned for reuse. A disposable diaper, for example, is discarded after it has become soiled by the wearer. Optionally, a disposable diaper may include a single-use, absorbent insert, and a limited-use outer cover that may be reused several times.

With regard to the designated surfaces of a disposable absorbent article and its components, the various upper or bodyfacing surfaces are configured to face toward the body of the wearer when the disposable absorbent article is worn by the wearer for ordinary use. The various opposing, lower or garment facing surfaces are configured to face away from the wearer's body when the disposable absorbent article is worn by the wearer.

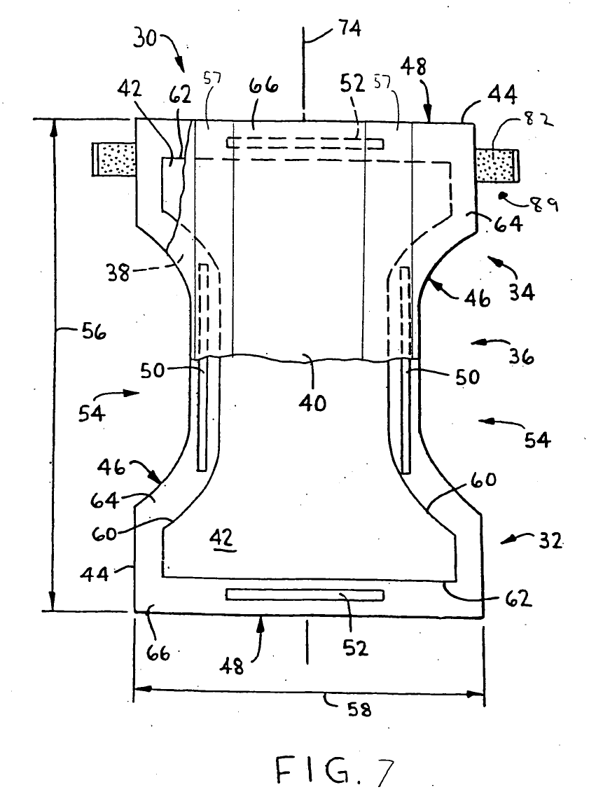

Referring now to the drawings, FIG. 7 illustrates a disposable absorbent article such as a disposable diaper (30) in an unfolded, flat-out, uncontracted state (i.e., with all elastic induced gathering and contraction removed). Portions of the structure are partially cut away to more clearly show the interior construction of the diaper (30), with the surface of the diaper (30) which contacts the wearer facing the viewer. FIG. 7 illustrates a disposable diaper (30) as having a front region (32), a rear region (34) and a crotch portion (36) located between the front and rear regions. The diaper (30) comprises a backsheet (38), a topsheet (40), and an absorbent core (42) situated between the backsheet and the topsheet. The outer edges of the diaper (30) define a periphery (44) with transversely opposed, longitudinally extending side edges (46); longitudinally opposed, transversely extending end edges (48); and a system of elastomeric gathering members, such as a system including leg elastics (50) and waist elastics (52). The longitudinal side edges (46) define the leg openings (54) for the diaper (30), and optionally, are curvilinear and contoured. The transverse end edges (48) are illustrated as straight, but optionally, may be curvilinear. The diaper (30) may also comprise additional components to assist in the acquisition, distribution and storage of bodily waste. For example, the diaper (30) may comprise a transport layer, such as described in U.S. Pat. No. 4,798,603, issued to Meyer et al., or a surge management layer, such as described in European Patent Application Publication No. 0 539 703, published May 5, 1993.

The diaper (30) generally defines a longitudinally extending length dimension (56), and a laterally extending width dimension (58), as representatively illustrated in FIG. 7. The diaper (30) may have any desired shape, such as rectangular, 1-shaped, a generally hourglass shape, or a T-shape.

The backsheet (38) defines a length and a width that, in the illustrated version, coincide with the length and width of the diaper (30). The absorbent core (42) generally defines a length and width that are less than the length and width of the backsheet (38), respectively. Thus, marginal portions of the diaper (30), such as marginal sections of the backsheet (38), may extend past the transversely opposed, longitudinally extending terminal side edges (60) and/or the longitudinally opposed, transversely extending terminal end edges (62) of the absorbent core (42) to form side margins (64) and end margins (66) of the diaper (30). The topsheet (40) is generally coextensive with the backsheet (38), but may optionally cover an area that is larger or smaller than the area of the backsheet, as desired. The backsheet (38) and topsheet (40) are intended to face the garment and body of the wearer, respectively, while in use. As used herein when describing the topsheet (40) in relation to the backsheet (38) and vice versa, the term “associated” encompasses configurations in which the topsheet is directly joined to the backsheet, and configurations where the topsheet is indirectly joined to the backsheet by affixing portions of the topsheet to intermediate members which in turn are affixed to at least portions of the backsheet. The topsheet (40) and the backsheet (38) can, for example, be joined to each other in at least a portion of the diaper periphery (44) by attachment mechanisms (not shown) such as adhesive bonds, sonic bonds, thermal bonds, pinning, stitching, or a variety of other attachment techniques known in the art, as well as combinations thereof.

The topsheet (40) suitably presents a bodyfacing surface which is compliant, soft feeling, and non-irritating to the wearer's skin. Further, the topsheet (40) may be less hydrophilic than the absorbent core (42), to present a relatively dry surface to the wearer, and is sufficiently porous to be liquid permeable, permitting liquid to penetrate readily through its thickness. A suitable topsheet (40) may be manufactured from a wide selection of web materials, such as porous foams, reticulated foams, apertured plastic films, natural fibers, synthetic fibers (for example, polyester or polypropylene fibers), or a combination of natural and synthetic fibers. The topsheet (40) is suitably employed to help isolate the wearer's skin from liquids held in the absorbent core (42).

Various woven and nonwoven fabrics may be used for the topsheet (40). For example, the topsheet (40) may be composed of a meltblown or spunbonded web of polyolefin fibers. The topsheet (40) may also be a bonded-carded web composed of natural and/or synthetic fibers. The topsheet (40) may be composed of a substantially hydrophobic material, and the hydrophobic material may, optionally, be treated with a surfactant, or otherwise processed, to impart a desired level of wettability and hydrophilicity. Specifically, the topsheet (40) may be a nonwoven, spunbond, polypropylene fabric composed of about 2.8 to about 3.2 denier fibers formed into a web having a basis weight of about 22 gsm and a density of about 0.06 g/cc.

The topsheet (40) may also be surface treated with about 0.3 weight percent of a surfactant mixture that contains a mixture of AHCOVEL Base N-62 surfactant and GLUCOPON 220UP surfactant in about a 3:1 ratio based on a total weight of the surfactant mixture. The AHCOVEL Base N-62 surfactant is purchased from Hodgson Textile Chemicals Inc., a business having offices in Mount Holly, N.C., and comprises a blend of hydrogenated ethoxylated castor oil and sorbitan monooleate in a 55:45 weight ratio. The GLUCOPON 220UP surfactant is purchased from Henkel Corporation, Gulph Mills, Pa., and comprises alkyl polyglycoside. The surfactant may also include additional ingredients such as aloe. The surfactant may be applied by any conventional means, such as spraying, printing, brush coating, foam or the like. The surfactant may be applied to the entire topsheet (40) or may be selectively applied to particular sections of the topsheet, such as the medial section along the longitudinal centerline of a diaper, to provide greater wettability of such sections.

The backsheet (38) may suitably be composed of a material which is either liquid permeable or liquid impermeable. It is generally desirable that the backsheet (38) be formed from a substantially liquid impermeable material. For example, a typical backsheet (38) can be manufactured from a thin plastic film or other flexible liquid impermeable material. Moreover, the backsheet (38) may be formed from a polyethylene film having a thickness of from about 0.012 mm (0.5 mil) to about 0.051 mm (2.0 mils). If desirous of presenting the backsheet (38) with a more cloth-like feel, the backsheet may comprise a polyethylene film having laminated to the lower or opposing surface thereof a nonwoven web, such as a spunbond web of polyolefin fibers. For example, a polyethylene film having a thickness of about 0.015 mm (0.6 mil) may have thermally laminated thereto a spunbond web of polyolefin fibers, which fibers have a thickness of about 1.5 to about 2.5 denier per filament, which nonwoven web has a basis weight of about 24 gsm (0.7 osy). Methods of forming such cloth-like outer covers are known to those skilled in the art. Further the backsheet (38) may be a stretchable material, a method of forming such a material may be found in U.S. Pat. No. 5,226,992 issued to Morman, further various examples of extensible materials are described in U.S. Pat. No. 6,264,641 issued to VanGompel et al.; the entire disclosures of which are hereby incorporated by reference in a manner that is consistent herewith

Further, the backsheet (38) may be formed of a woven or nonwoven fibrous web layer which has been totally or partially constructed or treated to impart a desired level of liquid impermeability to selected regions that are adjacent or proximate the absorbent core (42). Still further, the backsheet (38) may optionally be composed of micro-porous “breathable” material that permits vapors to escape from the absorbent core (42) while still preventing liquid exudates from passing through the backsheet.

The absorbent core (42) may comprise a matrix of hydrophilic fibers, such as a web of cellulosic fluff, mixed with particles of a high-absorbency material commonly known as superabsorbent material. In a particular version, the absorbent core (42) comprises a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The absorbent core (42) may have any of a number of shapes. For example, the absorbent core (42) may be rectangular, 1-shaped or T-shaped. It is often considered as desirable for the absorbent core (42) to be narrower in the crotch portion than the rear or front region(s).

The high-absorbency material can be selected from natural, synthetic and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble, but swellable. Such means can comprise, for example, physical entanglement, crystalline domains, covalent bonds, ionic complexes and associations, hydrophilic associations, such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al.

The high-absorbency material may be in a variety of geometric forms. It is desired that the high-absorbency material be in the form of discrete particles. However, the high-absorbency material may also be in the form of fibers, flakes, rods, spheres, needles, or the like. Often, the high-absorbency material is present in the absorbent core (42) in an amount of from about 5 to about 100 weight percent based on total weight of the absorbent core.

As representatively illustrated in FIG. 7, the diaper (30) may include a pair of containment flaps (57) that are configured to provide a barrier to the lateral flow of body exudates. The containment flaps (57) may be located along the longitudinally extending side edges (46) of the diaper (30) adjacent the side edges of the absorbent core (42). Each containment flap (57) typically defines an unattached edge that is configured to maintain an upright, perpendicular configuration in at least the crotch portion (36) of the diaper (30) to form a seal against the wearer's body. The containment flaps (57) may extend longitudinally along the entire length of the absorbent core (42) or may only extend partially along the length of the absorbent core (42). When the containment flaps (57) are shorter in length than the absorbent core (42), the containment flaps (57) can be selectively positioned anywhere along the side edges (46) of the diaper (30) in the crotch portion (36). In a particular aspect of the invention, the containment flaps (57) extend along the entire length of the absorbent core (42) to better contain the body exudates. Such containment flaps (57) may utilize the elastic composites (16) of the present invention.

The diaper (30) of the different configurations of the present invention may further include elastics at the end edges (48) and side edges (46) of the diaper (30) to further prevent leakage of body exudates and support the absorbent core (42). For example, as representatively illustrated in FIG. 1, the diaper (30) of the present invention may include a pair of leg elastics (50) that are connected to the side edges (46) of the diaper (30) crotch portion (36). The diaper (30) may also include a pair of waist elastics (52) that are connected to the end edges (48) of the diaper (30). The leg elastics (50) and waist elastics (52) are generally adapted to fit about the legs and waist of a wearer in use to maintain a positive, contacting relationship with the wearer to effectively reduce or eliminate the leakage of body exudates from the diaper (30).

The elastic composites (16) of the present invention are suitable for use as the leg elastics (50), waist elastics (52) or other elastic components. The elastic composites (16) may be adhered to the backsheet (38) in a stretched position, or that may be attached to the backsheet (38) while the backsheet (38) is pleated, such that elastic constrictive forces are imparted to the backsheet (38). The leg elastics (50) may also include such materials as polyurethane, synthetic and natural rubber. The waist elastics (52) may be formed by elastic strands attached to the backsheet (38) utilizing the present invention or they may be formed by attaching separate pieces of stretchable materials to the waist regions of the article wherein the separate pieces utilize the present invention. For example, the waist elastics 58 may include a piece of the elastic composite (16) attached to the interior surface of the article to form a waistband.

The disposable absorbent articles described herein also comprise fasteners (82) for securing the absorbent article about the waist of the wearer. The illustrated versions of the diaper (30) comprise such fasteners (82). In at least one version, the fasteners (82) are situated in the rear region (34) of the diaper (30), and are located inboard each longitudinal extending side edge (46). The fasteners (82) may be configured to encircle the hips of the wearer and engage the backsheet (38) of the front region (32) of the diaper (30) for holding the diaper (30) on the wearer. Suitable fasteners are well known to those of skill in the art and can comprise adhesive tape tab fasteners, hook and loop fasteners, mushroom fasteners, snaps, pin, belts and the like, and combinations thereof. Desirably, the fasteners (82) are releasably engageable directly with the garment-facing surface of the backsheet (38). Desirably, the fasteners (82) comprise a mechanical fastening system.

It will be appreciated that details of the elastic composites of the invention, given for purposes of illustration, are not to be construed as limiting the scope of this invention. Although only a few exemplary aspects of this invention have been described in detail above, those skilled in the art will readily appreciate that many modifications are possible in the exemplary aspects without materially departing from the novel teachings and advantages of this invention. Accordingly, all such modifications are intended to be included within the scope of this invention, which is defined in the following claims and all equivalents thereto. Further, it is recognized that many aspects may be conceived that do not achieve all of the advantages of some aspects, particularly of the preferred aspects, yet the absence of a particular advantage should not be construed to necessarily mean that such an aspect is outside the scope of the present invention.

What is claimed is:

1. An elastic composite comprising:
a first layer;
a second layer;
at least one elastic strand located between the first layer and the second layer and defining a width; and
a plurality of thermoplastic bonds joining the first and second layers, at least a portion of the thermoplastic bonds containing a segment of the elastic strand, the portions having a spacing perpendicular to the elastic strand that is less than the width of the elastic strand.

2. The elastic composite of claim 1 wherein the thermoplastic bonds that contain the elastic strand have a dimension perpendicular to the elastic strand that is less than the width of the elastic strand.

3. The elastic composite of claim 2 wherein the thermoplastic bonds that contain the elastic strand have a dimension perpendicular to the elastic strand that is greater than 0.15 and less than 0.9 times the width of the elastic strand.

4. The elastic composite of claim 2 wherein the spacing in a direction perpendicular to the elastic strand is less than 0.9 times the width of the elastic strand.

5. The elastic composite of claim 1 wherein the spacing in a direction perpendicular to the elastic strand is greater than 0.5 times the width of the elastic strand.

6. The elastic composite of claim 1 wherein the elastic strand has a cross sectional shape of a square.

7. The elastic composite of claim 1 comprising a plurality of elastic strands.

8. The elastic composite of claim 1 wherein the thermoplastic bonds intermittently bond the elastic strand to the first and second layers.

9. The elastic composite of claim 8 wherein the elastic composite has a first basis weight in an area containing the thermoplastic bonds, the elastic composite has a second basis weight in an area remote from the thermoplastic bonds and the first basis weight is greater than the second basis weight.

10. The elastic composite of claim 9 wherein the elastic composite in the area containing the thermoplastic bonds further comprises additional thermoplastic material.

11. The elastic composite of claim 10 wherein the additional thermoplastic material is chosen from the group of polyethylene, polypropylene, formulated polypropylene and formulated polyethylene.

12. The elastic composite of claim 1 wherein the first and second layers comprise portions of an integral material.

13. The elastic composite of claim 1 wherein the elastic strand has a decitex of less than 5000.

14. The elastic composite of claim 1 wherein the elastic strand comprises a plurality of elastic threads.

15. The elastic composite of claim 1 wherein the plurality of thermoplastic bonds are arranged in substantially parallel rows to provide a plurality of paths through which the elastic strand may pass.

16. A disposable absorbent article comprising:
an absorbent core;
an outer cover positioned adjacent the absorbent core; and
a pair of containment flaps extending at least partially along the length of the absorbent core, the containment flaps comprising:
a first layer;
a second layer;
at least one elastic strand located between the first layer and the second layer and defining a width; and
a plurality of thermoplastic bonds joining the first and second layers, at least a portion of the thermoplastic bonds containing a segment of the elastic strand, the portions having a spacing perpendicular to the elastic strand that is less than the width of the elastic strand.

17. The disposable absorbent article of claim 16 wherein the thermoplastic bonds that contain the elastic strand have a dimension perpendicular to the elastic strand that is less than the width of the elastic strand.

18. The disposable absorbent article of claim 16 wherein the thermoplastic bonds that contain the elastic strand have a dimension perpendicular to the elastic strand that is greater than 0.15 and less than 0.9 times the width of the elastic strand.

19. The disposable absorbent article of claim 16 wherein the spacing in a direction perpendicular to the elastic strand is less than 0.9 times the width of the elastic strand.

20. The disposable absorbent article of claim 16 wherein the thermoplastic bonds intermittently bond the elastic strand to the first and second layers.

21. The disposable absorbent article of claim 20 wherein the containment flaps have a basis weight in an area containing the thermoplastic bonds, the elastic composite has a basis weight in an area remote from the thermoplastic bonds and the basis weight in the area containing the thermoplastic bonds is greater than the basis weight in the area remote from the thermoplastic bonds.

22. The disposable absorbent article of claim 21 wherein the containment flaps in the area containing the thermoplastic bonds further comprises additional thermoplastic material.

23. The disposable absorbent article of claim 22 wherein the additional thermoplastic material is chosen from the group of polyethylene, polypropylene, formulated polypropylene and formulated polyethylene.

24. The disposable absorbent article of claim 16 wherein the first and second layers comprise portions of an integral material.

25. The disposable absorbent article of claim 16 wherein the elastic strand has a decitex of less than 2000.

26. The disposable absorbent article of claim 16 wherein the plurality of thermoplastic bonds are arranged in substantially parallel rows to provide a plurality of paths through which the elastic strand may pass.

27. An elastic composite comprising:
a first layer;
a second layer at least one elastic strand located between the first and second layers; and
a plurality of thermoplastic bonds joining the first layer and the second layer;
wherein portions of the elastic strand are contained in at least a portions of the thermoplastic bonds, the elastic strand has a width, and the thermoplastic bonds containing the elastic strand have a spacing in a direction perpendicular to the elastic strand that is between 0.1 and 0.9 times the width of the elastic strand, the thermoplastic bonds containing the elastic strand have a dimension perpendicular to the elastic strand that is between 0.2 and 0.6 times the width of the elastic strand, the elastic strand has a cross sectional shape of a circle, the thermoplastic bonds intermittently bond the elastic strand to the first and second layers, and the elastic strand has a decitex of less than 2000.

28. The elastic composite of claim 27 wherein the elastic composite has a first basis weight in an area containing the thermoplastic bonds, the elastic composite has a second basis weight in an area remote from the thermoplastic bonds and the first basis weight is greater than the second basis weight.

29. The elastic composite of claim 27 wherein the elastic composite in the area containing the thermoplastic bonds further comprises additional thermoplastic material.

30. The elastic composite of claim 29 wherein the additional thermoplastic material located in the area containing the thermoplastic bonds is not located in the area remote from the thermoplastic bonds.

31. The elastic composite of claim 30 wherein the additional thermoplastic material is chosen from the group of polyethylene, polypropylene, formulated polypropylene and formulated polyethylene.

32. A method of producing an elastic composite comprising
providing a first layer;
providing a second layer;
providing at least one elastic strand located between the first layer and the second layer and defining a width; and
joining the first and second layers with a plurality of thermoplastic bonds, at least a portion of the thermoplastic bonds containing a segment of the elastic strand, the portions having a spacing perpendicular to the elastic strand that is less than the width of the elastic strand.

33. The method of claim 32 wherein the plurality of thermoplastic bonds are arranged in substantially parallel rows to provide a plurality of paths through which the elastic strand may pass.
